(19) United States
(12) Patent Application Publication
(10) Pub. No.: US 2011/0183935 A1
(43) Pub. Date: Jul. 28, 2011

(54) HEARTWATER

(76) Inventor: Aaron Feingold, Morganville, NJ (US)

(21) Appl. No.: 13/013,477

(22) Filed: Jan. 25, 2011

Related U.S. Application Data
(60) Provisional application No. 61/297,957, filed on Jan. 25, 2010.

(51) Int. Cl. A61K 31/716; A61K 31/56; A61K 31/202; A61P 9/00; A61P 3/06

(52) U.S. Cl. 514/54; 514/169; 514/560

(57) ABSTRACT

There is provided a water product for general consumer comprising water suitable for human consumption and a food additive in the amount effective to alter a level of serum marker that indicates a likelihood of cardiovascular disease, thereby the water product is intended to lower the risk of cardiovascular disease of said general consumer. The preferred additive is Barliv™ betafiber.

### CROSS-REFERENCE TO RELATED APPLICATIONS

This application claims the benefit of U.S. Provisional Patent Application Ser. No. 61/297,957, filed on Jan. 25, 2010, the entirety of the disclosure of which application is incorporated herein by reference.

### FIELD OF THE INVENTION

The present invention, HEARTWATER, pertains to a drink that promotes health heart by reducing levels of serum cholesterol in humans, and more particularly to a water-based drink containing a beta-glucan that can reduce cholesterol.

### BACKGROUND

Heart disease is the number one cause of human deaths in the United States. According to the American Heart Association, 48% of American adults have elevated levels of serum cholesterol, i.e., >200 mg/dL, which is a leading contributing factor to cardiovascular disease.

Presently, persons who have high cholesterol can lower their cholesterol through a combination of diet and exercise. For example, eating foods that are low in saturated fats and include a beta-glucan, together with regular exercise, can help lower cholesterol. However, diet and exercise alone are often times unable to lower a person's cholesterol. Alternatively, many persons with high cholesterol take prescription drugs such as statins to lower their cholesterol. Although statins are widely prescribed, many persons are unable to take them because of the side effects, e.g., severe joint pain, often associated with their use.

It is therefore an object of the present invention to provide an enhanced water-based drink that is a safe, easy to use alternative and/or adjunct to the aforementioned conventional treatments that are conventionally used to lower serum cholesterol. In addition, it is an object of the present invention to provide an enhanced water-based drink about which a cardiovascular-related health claim can be made that has been approved by the U.S. Food and Drug Administration (“FDA”).

### SUMMARY

In one aspect, the invention provides a water product for a general consumer comprising water suitable for human consumption and a food additive in the amount effective to alter a level of serum marker that indicates a likelihood of cardiovascular disease, thereby said water product is intended to lower the risk of cardiovascular disease of said general consumer. Preferably, the water product is not carbonated. In one variant of this aspect of the invention, the water product does not contain a sweetener.

The water product is intended to improve general cardiovascular health of general consumer consuming the water product by targeting and altering the level of serum markers associated with cardiovascular health. The preferred serum market is LDL cholesterol.

One preferred food additive is a beta-glucan. Non-limiting examples of beta-glucans include beta glucans made from oat bran, rolled oats, whole oat flour, or whole grain barley. The preferred beta-glucan is Barliv™ beta fiber made by Cargill. In another variant of this aspect of the invention, the food additive is plant sterol or Omega 3 oil.

The water product of the invention may include an organic acid suitable for human consumption. Non-limiting examples of the organic acids include citric acid, malic acid, and tartaric acid.

The water product of the invention may include a flavorant. Preferably, the flavorant is a natural flavorant.

The water product of the invention may also contain one or more ingredients that satisfy the Jelly Bean Rule in the amount sufficient to satisfy the rule.

The water product of the invention may also include one or more of a taste modifier, a vitamin, a mineral, a buffering agent, a colorant, an antioxidant, or a preservative.

Preferably, the food additive may be present in the amount ranging from 0.1% to 3% w/v, more preferably, in the amount ranging from 0.3% to 1% w/v, yet more preferably, in the amount ranging from 0.35% to 0.6% w/v.

Preferably, Barliv™ beta fiber is present in the amount ranging from 0.3% to 1%, w/v, with respect to the total volume of said water product.

Preferably, the organic acid present in the amount ranging from 0.1% to 0.5%, w/v, with respect to the total volume of said water product.

Preferably, the natural flavorant present in the amount ranging from 0.1% to 0.5%, w/v, with respect to the total volume of said water product.

Preferably, the water product includes a natural sweetener present in the amount ranging from 1% to 8%, w/v, with respect to the total volume of said water product.

In the preferred embodiment, the invention provides a water product for general consumer comprising water suitable for human consumption and a) 0.1% to 0.6% of Barliv™ beta fiber, b) 0.1 to 0.3% of malic acid, and c) 4% to 7% of agave syrup, with respect to the total volume of said water product.

In another aspect, the invention provides a method of improving cardiovascular health of a general consumer comprising administering the water product of the first aspect of the invention to the general consumer.

### DETAILED DESCRIPTION

The present invention is defined by the appended claims. The terms used in the claims are defined as follows.

The term “water product” defines a water-based product similar to spring water sold to the general consumer and does not encompass any water-based product containing a fruit or vegetable juice. Preferably, the water product of the invention is not carbonated.

The term “serum markers associated with cardiovascular health” denotes any well-known protein or other endogenous biomolecule the level of which in a human is associated with the likelihood of adverse cardiovascular events or improved cardiovascular health. Examples of such serum markers include LDL cholesterol and HDL cholesterol. Thus, it is known that elevated level of LDL cholesterol is associated with the likelihood of adverse cardiovascular event. In contrast, a rise in the level of HDL cholesterol within parameters accepted within the medical profession is associated with improvement in cardiovascular health.

The term “altering the level of” with respect to food additives that effect the serum marker defines any statistically significant change in the level of serum marker, preferably, change in the level of the serum marker exceeding 5%.

The term “food additive” is used to denote a substance suitable for human consumption if included in a water-containing or food product for consumption by a general consumer. Examples of food additives suitable for the use with the present invention include Barliv™ beta fiber, plant styrol, and Omega 3 oil.

The present invention provides an enhanced water product targeted to improve the cardiovascular health of a general consumer. In particular, the invention provides a water product intended to be consumed in the same manner as the spring water routinely sold through consumer outlet, improving cardiovascular health without interfering with daily functioning of the consumer. In addition to water, the product includes a food additive in the amount sufficient to alter the level of serum markers associated with cardiovascular health. In particular, the suitable water product is preferably intended to reduce the risk of cardiovascular disease by lessening specific factors that have been shown to statistically increase the likelihood of cardiovascular disease. Many blood serum markers, e.g., homocysteine protein, c reactive protein, indicate this higher risk of cardiovascular disease. The present invention targets these markers, and lessens, neutralizes, blocks their absorption of, or lowers the pathogenesis of these markers, to lower the risk of cardiovascular disease. For example, a serum marker affected by the present invention is low density lipid protein, i.e., LDL cholesterol. By lowering LDL cholesterol, the present invention lowers the risk and incidence of cardiovascular disease.

The preferred food additive of the present invention is beta-glucan. Beta-glucans are polysaccharides containing glucose monomer units which are bonded by .beta.-linkages. D-glucose is the naturally occurring isomer of glucose. Suitable beta-glucans may be derived from grains, such as oats, barley, rye, and wheat. Specifically, beta-glucan may be derived from oat bran, rolled oats, whole oat flour, oatrim, whole grain barley, and dry milled barley. The particularly preferred beta-glucan is Barliv™ beta fiber manufactured by Cargill. It has been shown in clinical studies that consumption of beta-glucan derived from barley fiber, particularly, Barliv™ beta fiber, reduces the risk of coronary heart disease via reductions in the levels of LDL cholesterol. Preferably, the water product of the invention includes beta glucan in the amount ranging from 0.1% to 3%, more preferably, from 0.3% to 1%, yet more preferably, from 0.35% to 0.6%, w/v, based on the total volume of the water product.

The water product described herein may further include a sweetener. However, an embodiment of the water product which does not include a sweetener is specifically contemplated. The preferred sweeteners suitable for use with water product of the present invention are natural sweeteners. Suitable sweeteners may be selected and optimized for the desired characteristics, taste profile, beverage mouth feel and other similar factors. Natural sweeteners suitable for use with the water product of the invention include agave syrup, erythritol, tagatose, sorbitol, mannitol, xylitol, maltose, rhamnose, trehalose, glycyrrhizin, malitol, lactose, Lo Han Guo (“LHG”), rebaudiosides, steviol glycosides, xylose, arabinose, isomalt, lactitol, maltitol, and ribose, and protein sweeteners such as thaumatin, monellin, brazzein, and monatin. The sweetener component of the water product may be used as a taste masking agent, particularly, when the food additive present in the effective amount has generally unpleasant taste. Generally, the preferred amount of sweetener ranges from 1% to 8% based on the total volume of the water product, although larger or smaller concentrations may be used if desired. The amount of sweetener used to taste-mask may be selected based on the tasting protocols.

The water product may also include various additional ingredients, such as other sweeteners, vitamins, minerals, flavoring agents, acidulants, coloring agents, emulsifiers, thickeners, stabilizers, pH adjusters, preservatives, carbonation, and caffeine, among others. Other additives, such as those described hereinabove, are also contemplated and within the scope of the invention.

The water product may include at least one flavoring agent. Flavoring agents may be, for example, fruit flavors, botanical flavors, spice flavors, and taste modifiers, among others. The use of flavorants allows for creation of water products of different flavors, e.g., mango, orange, etc.

The water product may include a variety of taste modifiers to reduce or mask undesirable taste of another ingredient, for example a food additive. Non-limiting examples of undesirable taste characteristics include bitter aftertaste, metallic aftertaste, astringency, thin mouth feel, harshness, delayed sweetness onset, lingering sweetness, and excess sourness. Non-limiting examples of desirable taste characteristics include sweetness (when desired), fullness, and smoothness. Non-limiting examples of taste modifiers include organic acids (e.g., citric acid, malic acid, ascorbic acid, tartaric acid, lactic acid, adipic acid, fumaric acid, gluconic acid, succinic acid, maleic acid, among others), propylene glycol, glycerol, and ethanol.

The flavorant component of the water product may be a suitable fruit flavor, which may be any fruit fraction, fruit component (e.g., rind, zest, pith, pericarp, pulp, flower (e.g., petals), leaf, stem, and seed), from the named fruit (FTNF) flavor (e.g., a combination of fruit essence, fruit oil and/or fruit flavor, such as, e.g., an orange from the named fruit flavor), fruit extract, fruit oil (e.g., essential oil, folded essential oil), fruit essence, fruit puree, and fruit aroma. In certain exemplary embodiments, one or more citrus fruit flavors are used. The citrus flavor may include one or more of an orange fraction, an orange component, an orange extract, an orange essential oil, an orange folded essential oil, an orange aroma, and an orange essence. The citrus flavor may also include one or more of a fraction, component, extract, essential oil, folded essential oil, aroma, or essence of grapefruit, lemon, lime, or tangerine, among others. The citrus flavor may also include chemical compounds extracted from natural sources or synthetically produced e.g., limonene, octanol and its derivatives, acetaldehyde, .alpha.-pinene, .beta.-pinene, sabinene, myrcene, octanal, linalool, carene, decanal, citral, and sinensal.

The water product may include an organic acid suitable for human consumption in a water or food product. The organic acid may be used to improve the taste of the water product, enhance palatability, increase thirst quenching effect, etc. Non-limiting examples of organic acids include citric acid, malic acid, ascorbic acid, tartaric acid, lactic acid, adipic acid, fumaric acid, gluconic acid, succinic acid, and maleic acid.

It is contemplated that the water product of the invention may include one or more added ingredient to comply with the FDA's Jelly Bean Rule, such as vitamins, e.g., Vitamin A, Vitamin B1 (i.e., thiamine), Vitamin B2, Vitamin B. 3, Vitamin B6, Vitamin B12 (cobalamin), Vitamin C, Vitamin D, Vitamin E, and folic acid. The water product may also contain one or more added minerals, e.g., added calcium, potassium, magnesium, phosphorous, zinc, and iron, among others. Certain exemplary embodiments of the beverages disclosed here also may contain small amounts of buffering agents to adjust pH. Such pH adjusters include, e.g., the sodium and potassium salts of citric, tartaric, and lactic acids. The amount included will depend, of course, on the type of buffering agents and on the degree to which the pH is to be adjusted. The Jelly Bean Rule says foods low in fat, cholesterol, and sodium cannot claim to be “healthy” unless they contain at least 10 percent of the Recommended Daily Allowance (“RDA”) of: vitamin A; vitamin C; calcium; protein; fiber; or iron. See http://www.cnn.com/HEALTH/9512/food_labels/index.html, incorporated herein by reference.

The preparation of the water product of the invention may be accomplished by providing the water and then adding the desired ingredient in any manner, including a manner known in the art of beverages.

The present invention is illustrated in the appended non-limiting Examples.

### Example 1

### Heart Water-40 Calories

Example 1 provides an embodiment of the water product of the invention.

| Purified Water | QS to 1000 | mls |
|---|---|---|
| Agave Syrup Light | 60.00 | g |
| Malic Acid | 1.40 | g |
| Barliv | 4.57 | g |
| Omega Cran | 0.02 | g |
| Coenzyme Q10 | 0.03 | g |
| Fortitech Vitamin Blend (FT072396) | 0.03 | g |
| Acerola Puree | 2.60 | g |
| Natural Peach Mango Fl | @0.14% | v/v |
| Sensient Beta Carotene 2% | @0.03 | g/liter |
| Natural Lemon Flavor | @0.12% | v/v |

### Example 2

Example 2 provides another embodiment of the water product of the invention.

| Purified Water | QS to 1000 | mls |
|---|---|---|
| Agave Syrup | 60.00 | g |
| Malic Acid | 1.40 | g |
| Cargill Barliv | 4.57 | g |
| Acerola Puree | 2.60 | g |
| 271662 Natural Watermelon Yumberry | 0.11% | v/v |
| Flavor WONF | | |
| GNT Royal Pink Natural Color | 0.005% | v/v |

### Brix: 7.0 pH: 2.9

Approximately per 8 oz serving: Calories—40, Barliv 1081 mg, Acerola 615 mg

### Example 3

Example 3 provides another embodiment of the water product of the invention.

| Purified Water | QS to 1000 | mls |
|---|---|---|
| Agave Syrup | 60.00 | g |
| Malic Acid | 1.40 | g |
| Cargill Barliv | 4.57 | g |
| Acerola Puree | 2.60 | g |
| 271663 Natural Pomegranate | @0.12% | v/v |
| Blueberry Flavor WONF | | |
| GNT Bordeaux Natural Color | @0.01% | v/v |

### Brix: 7.0 pH: 2.9

Approximately per 8 oz serving: Calories—40, Barliv 1081 mg, Acerola 615 mg

### Example 4

Example 4 provides another embodiment of the water product of the invention.

| Purified Water | QS to 1000 | mls |
|---|---|---|
| Agave Syrup | 60.00 | g |
| Malic Acid | 1.40 | g |
| Cargill Barliv | 4.57 | g |
| Acerola Puree | 2.60 | g |
| 271664 Natural Tropical Punch | @0.11% | v/v |
| Flavor | | |
| Sensient Beta Carotene 2% | @0.002% | v/v |

### Brix: 7.0 pH: 2.9

Approximately per 8 oz serving: Calories—40, Barliv 1081 mg, Acerola 615 mg

### Example 5

### Heart Water-Pomegranate/Blueberry

Example 5 provides another embodiment of the water product of the invention.

| Agave Syrup | 67.50 | g |
|---|---|---|
| Malic Acid | 1.67 | g |
| Wild Phytosterol Esters | 15.80 | g |
| 17% FAIZ906 | | |
| Omega 3 Oil | 0.42 | g |
| CoQ10 | 0.029 | g |
| Fortitech Vitamin Premix | 0.025 | g |
| FT072396 | | |
| Exberry Color Shade | 0.15 | g |
| “Fushia” | | |
| Natural Pomegranate | 0.10% | |
| Flavor #180626 | | |
| Natural Blueberry | 0.10% | |
| Flavor #180331 | | |
| Purified Water | To Volume | |
| | 1000 | mL |

### Example 6

### Heart Water-Pineapple

Example 6 provides another embodiment of the water product of the invention.

| Agave Syrup | 67.50 | g |
|---|---|---|
| Malic Acid | 1.67 | g |
| Wild Phytosterol Esters | 15.80 | g |
| 17% FAIZ906 | | |
| Omega 3 Oil | 0.42 | g |
| CoQ10 | 0.029 | g |
| Fortitech Vitamin Premix | 0.025 | g |
| FT072396 | | |
| Natural Coconut Flavor | 0.08% | |
| #180690 | | |
| Natural Pineapple | 0.12% | |
| Flavor #180323 | | |
| Purified Water | To Volume | |
| | 1000 | mL |

### Example 7

Example 7 provides another embodiment of the water product of the invention.

| | |
|---|---|
| 1000 | mls of Purified Water |
| 4.57 | grams of Barliv |

### Example 8

Example 8 provides another embodiment of the water product of the invention.

| | |
|---|---|
| 1000 | mls of Purified Water |
| 4.57 | grams of Barliv |
| 2.60 | grams of Acerola Puree |

### Example 9

Example 9 provides another embodiment of the water product of the invention.

| | |
|---|---|
| 1000 | mls of Purified Water |
| 4.57 | grams of Barliv |
| 2.60 | grams of Acerola Puree |
| 60.00 | grams of Agave Syrup |

Numerous modifications to and alternative embodiments of the present invention will be apparent to those skilled in the art in view of the foregoing description. Accordingly, this description is to be construed as illustrative only and is for the purpose of teaching those skilled in the art the best mode of carrying out the invention. Details of the embodiment may be varied without departing from the spirit of the invention, and the exclusive use of all modifications is hereby reserved.

We claim:

1. A water product for general consumer comprising water suitable for human consumption and a food additive in the amount effective to alter a level of serum marker that indicates a likelihood of cardiovascular disease, thereby said water product is intended to lower the risk of cardiovascular disease of said general consumer.

2. The water product of claim 1, which is not carbonated.

3. The water product of claim 2, which does not contain a sweetener.

4. The water product of claim 2, wherein said serum marker is LDL cholesterol.

5. The water product of claim 4, wherein said food additive is a beta-glucan.

6. The water product of claim 5, wherein said beta-glucan is made from oat bran, rolled oats, whole oat flour, or whole grain barley.

7. The product of claim 5, wherein the beta-glucan is Barliv™ beta fiber.

8. The water product of claim 4, wherein said food additive is plant sterol.

9. The water product of claim 1, further comprising organic acid suitable for human consumption.

10. The water product of claim 9, wherein said organic acid is selected from the group consisting of citric acid, malic acid, and tartaric acid.

11. The water product of claim 1, further comprising a flavorant.

12. The water product of claim 11, wherein said flavorant is a natural flavorant.

13. The water product of claim 1, further comprising one or more ingredients that satisfy the Jelly Bean Rule in the amount sufficient to satisfy said rule.

14. The water product of claim 1, further comprising one or more of a taste modifier, a vitamin, a mineral, a buffering agent, a colorant, an antioxidant, or a preservative.

15. The water product of claim 4, wherein said food additive is Omega 3 oil.

16. The water product of claim 1, wherein said food additive is present in the amount ranging from 0.1% to 3% w/v.

17. The water product of claim 16, wherein said food additive is present in the amount ranging from 0.3% to 1% w/v.

18. The water product of claim 16, wherein said food additive is present in the amount ranging from 0.35% to 0.6% w/v.

19. A method of improving cardiovascular health of a general consumer comprising administering the water product of claim 1 to said general consumer.

20. The method of claim 19, wherein said water product is administered on administration schedule selected by said general consumer.

21. The water product of claim 1, wherein said Barliv™ beta fiber is present in the amount ranging from 0.3% to 1%, w/v, with respect to the total volume of said water product.

22. The water product of claim 21, further comprising organic acid present in the amount ranging from 0.1% to 0.5%, w/v, with respect to the total volume of said water product.

23. The water product of claim 21, further comprising a natural flavorant present in the amount ranging from 0.1% to 0.5%, w/v, with respect to the total volume of said water product.

24. The water product of claim 21, further comprising a natural sweetener present in the amount ranging from 1% to 8%, w/v, with respect to the total volume of said water product.

25. A water product for general consumer comprising water suitable for human consumption and a) 0.1% to 0.6% of Barliv™ beta fiber, b) 0.1 to 0.3% of malic acid, and c) 4% to 7% of agave syrup, with respect to the total volume of said water product.

26. A method of improving cardiovascular health of a general consumer comprising administering the water product of claim 25 to said general consumer.

27. The method of claim 26, wherein said water product is administered on administration schedule selected by said general consumer.
